(19) United States
(12) Patent Application Publication
(10) Pub. No.: US 2011/0244484 A1
(43) Pub. Date: Oct. 6, 2011

(54) METHOD OF DIAGNOSING ALZHEIMER'S DISEASE USING GIANT MAGNETORESISTANCE SENSOR AND MAGNETIC BEAD-POLYPROTEIN COMPLEX FOR DIAGNOSING ALZHEIMER'S DISEASE

(75) Inventors: Cheol-Joo CHAE, Daejeon (KR); Kwan-Su Kim, Seoul (KR); Jae-Min Kang, Seoul (KR); Jeong-Dae Suh, Daejeon (KR); Myung-Ae Chung, Daejeon (KR); Ki-Bong Song, Daejeon (KR)

(73) Assignee: ELECTRONICS AND TELECOMMUNICATIONS RESEARCH INSTITUTE, Daejeon-city (KR)

(21) Appl. No.: 13/078,312

(22) Filed: Apr. 1, 2011

(30) Foreign Application Priority Data
Apr. 5, 2010 (KR) 10-2010-0030815
Oct. 4, 2010 (KR) 10-2010-0096495

(51) Int. Cl. G01N 33/53; C07K 14/36; C07K 14/47

(52) U.S. Cl. 435/7.21; 530/400

(57) ABSTRACT

Provided are a method of diagnosing Alzheimer's disease using a giant magnetoresistance sensor and a magnetic bead-polyprotein complex for diagnosing Alzheimer's disease. The method of diagnosing Alzheimer's disease using the giant magnetoresistance sensor may be applied to diagnose Alzheimer's disease more easily and simply using the giant magnetoresistance sensor than using conventional fluorescent materials or genetic analyses, and the magnetic bead-polyprotein complex may be mass-produced as a diagnostic biosensor for Alzheimer's disease, and thus to be useful to monitor and treat Alzheimer's disease.

### CLAIM FOR PRIORITY

This application claims priority to Korean Patent Applications Nos. 2010-0030815 filed on Apr. 5, 2010 and 2010-0096495 filed on Oct. 4, 2010, in the Korean Intellectual Property Office (KIPO), the entire contents of which are hereby incorporated by reference.

### BACKGROUND

1. Technical Field

Example embodiments of the present invention relate in general to a method of diagnosing Alzheimer's disease using a giant magnetoresistance sensor and a magnetic bead-polyprotein complex for diagnosing Alzheimer's disease.

2. Related Art

Alzheimer's disease is the leading cause of senile dementia and the most common degenerative neurological disease. Progressive Alzheimer's disease is characterized by loss of memory, and degeneration of linguistic ability, circumstantial judgement and reasonability. Generally, the characteristics of symptoms confused with physiological diseases caused at an advanced age, the severity of these symptoms, and the age at which these symptoms are caused are different in every individual. Therefore, exact etiological factors or therapeutic methods of Alzheimer's disease have not been known, and thus it is difficult to early diagnose Alzheimer's disease.

Representative pathological characteristics of Alzheimer's disease include senile plaques, neurofibrilary tangles, and neuronal loss. From many kinds of experimental evidence, aggregated amyloid-β protein which mainly appears to be the senile plaques is considered a major etiological factor of Alzheimer's disease.

Meanwhile, various methods of diagnosing Alzheimer's disease are known. Korean Patent Application Publication No. 10-2009-0048192 discloses therapeutic agents for diagnosis, prevention and treatment of dementia (Alzheimer's disease) and methods of screening the therapeutic agents. More particularly, the patent discloses that Alzheimer's disease may be diagnosed, prevented and treated using a binding inhibitor selected from the group consisting of FcγRIIb and a variant thereof, an FcγRIIb extracellular domain protein, an anti-FcγRIIb antibody, an FcγRIIb-specific peptide, and FcγRIIb-specific siRNA, the binding inhibitor relieving signal transduction by FcγRIIb and Aβ, intracellular transportation, neurotoxicity, apoptosis (programmed cell death) and memory impairments.

Also, Korean Patent Application Publication No. 10-2007-0073778 discloses that a technique of measuring β-amyloid in a biological sample (i.e., a specimen) such as blood is learned and applied to diagnosis of Alzheimer's disease. Also, it discloses a method capable of assaying Alzheimer's disease through immunoassay using an antibody recognizing a C-terminal domain of β-amyloid 1-42. Here, the method is performed by measuring the total amount of β-amyloid 1-42 fragments including β-amyloid 1-42 and a C-terminal domain of the β-amyloid 1-42 in the biological sample.

Furthermore, Korean Patent Application Publication No. 10-2009-0098941 discloses methods and compositions which may be used to detect Alzheimer's disease in mammals, especially humans. In particular, it discloses serum markers and methods used in diagnostic procedures for Alzheimer's disease. These methods are difficult to be commercially available because real-time diagnosis is difficult due to the use of an optical or chemical technique, the diagnosis costs are high, and it is impossible to mass-produce a kit for diagnosing Alzheimer's disease.

### SUMMARY

Accordingly, example embodiments of the present invention are provided to substantially obviate one or more problems due to limitations and disadvantages of the related art.

Example embodiments of the present invention provide a method of detecting Alzheimer's disease more simply and easily than conventional methods by detecting an amyloid-β protein using a giant magnetoresistance sensor.

Example embodiments of the present invention also provide a magnetic bead-polyprotein complex for diagnosing Alzheimer's disease by pretreating a magnetic bead used in diagnosis of Alzheimer's disease using a giant magnetoresistance sensor to be bound to a protein biomarker.

Example embodiments of the present invention also provide a method of preparing a magnetic bead-polyprotein complex for diagnosing Alzheimer's disease, the method including binding a magnetic bead used in diagnosis of Alzheimer's disease using a giant magnetoresistance sensor to a polyprotein so as to be bound to a protein biomarker.

In some example embodiments, a method of diagnosing Alzheimer's disease using a giant magnetoresistance sensor includes: pretreating a magnetic bead so that the magnetic bead can bind to a protein biomarker that causes Alzheimer's disease; arranging a cell to be diagnosed on the giant magnetoresistance sensor; disposing the pretreated magnetic bead on (above) the cell to be diagnosed, applying an external magnetic field to magnetize the pretreated magnetic bead; and detecting a change in magnetic field according to the binding of the pretreated magnetic bead to the protein biomarker using the giant magnetoresistance sensor and determining the presence of Alzheimer's disease in the cell to be diagnosed.

In this case, the change in magnetic field may be caused by a stray field generated in the magnetic bead when the protein biomarker is present and bound to the magnetic bead.

The protein biomarker may be an amyloid-β protein.

The magnetic bead may have a diameter of 50 nm to 5 μm.

The pretreatment may be performed by coating the magnetic bead with streptavidin, binding biotin to the streptavidin-coated magnetic bead, and binding poly(ethylene glycol) to the biotin.

The magnetoresistance sensor may use an anisotropic magnetoresistance thin film, a giant magnetoresistance thin film, a tunnel-type magnetoresistance thin film, and so on.

The magnetoresistance sensor may include a free layer, a spacer, a pinned layer and a pinning layer.

The magnetoresistance sensor may be in a cross or rod shape.

The magnetoresistance sensor may be passivated with an oxide or nitride thin-film layer.

The cell to be diagnosed may be a brain cell, an olfactory cell, a taste cell and a visual cell, in which the amyloid-β protein can accumulate.

The magnetization may be performed by applying the external magnetic field vertically or horizontally with respect to a surface of the giant magnetoresistance sensor.

In other example embodiments, a magnetic bead-polyprotein complex for diagnosing Alzheimer's disease is provided, in which the magnetic bead is coated with streptavidin, and the streptavidin is bound to biotin, the biotin being bound to a linker to be bound to a protein biomarker.

In this case, the protein biomarker may be an amyloid-β protein.

The linker used herein may include poly(ethylene glycol), and so on.

In other example embodiments, a method of preparing a magnetic bead-polyprotein complex for diagnosing Alzheimer's disease includes: coating a magnetic bead with streptavidin; binding biotin to the streptavidin-coated magnetic bead; and binding a linker for binding to a protein biomarker to the biotin.

### BRIEF DESCRIPTION OF DRAWINGS

Example embodiments of the present invention will become more apparent by describing in detail example embodiments of the present invention with reference to the accompanying drawings, in which:

FIG. 1 is a flowchart illustrating a method of diagnosing Alzheimer's disease using a giant magnetoresistance sensor according to one exemplary embodiment of the present invention.

FIG. 2 is a flowchart illustrating a method of preparing a magnetic bead-polyprotein complex for diagnosing Alzheimer's disease according to one exemplary embodiment of the present invention.

FIG. 3 is a schematic view illustrating a giant magnetoresistance sensor used to diagnose Alzheimer's disease according to one exemplary embodiment of the present invention.

FIG. 4 is a schematic view illustrating a structure of a giant magnetoresistance thin film used to diagnose Alzheimer's disease according to one exemplary embodiment of the present invention.

FIG. 5 is a schematic view illustrating a magnetic field direction of a magnetic bead when the magnetic bead is magnetized vertically with respect to a surface of the giant magnetoresistance sensor.

FIG. 6 is a schematic view illustrating a magnetic field direction of the magnetic bead when the magnetic bead is magnetized horizontally with respect to the surface of the giant magnetoresistance sensor.

FIG. 7 is a schematic view illustrating the magnetic bead-polyprotein complex for diagnosing Alzheimer's disease according to one exemplary embodiment of the present invention.

FIG. 8 is a schematic view illustrating a method of diagnosing Alzheimer's disease using a giant magnetoresistance sensor according to another exemplary embodiment of the present invention.

### DESCRIPTION OF EXAMPLE EMBODIMENTS OF THE PRESENT INVENTION

Example embodiments of the present invention are disclosed herein. However, specific structural and functional details disclosed herein are merely representative for purposes of describing example embodiments of the present invention, however, example embodiments of the present invention may be embodied in many alternate forms and should not be construed as limited to example embodiments of the present invention set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit is and scope of the invention. Like numbers refer to like elements throughout the description of the figures.

For the method of diagnosing Alzheimer's disease using a giant magnetoresistance sensor according to one exemplary embodiment of the present invention, a magnetic bead is essentially pretreated to be bound to a protein biomarker that causes Alzheimer's disease.

In this case, the protein biomarker may include an amyloid-β protein, and the protein biomarker may use cells present in a surface thereof.

Also, a pretreatment process is performed by coating the magnetic bead with streptavidin to selectively bind to an amyloid-β protein, which is known to cause Alzheimer's disease, selectively binding a biotin to the streptavidin-coated magnetic bead, and binding an amyloid-β protein to the biotin using a linker such as poly(ethylene glycol). A diameter of the magnetic bead may be in a range of 50 nm to 5 μm. When the diameter of the magnetic bead is less than 50 nm, it is difficult to sense a stray field generated in the magnetic bead, and when the diameter of the magnetic bead exceeds 5 μm, the magnetic bead cannot bind to amyloid-β present on a cell surface.

The pretreatment provides the magnetic bead to selectively bind to the amyloid-β protein. That is, since the amyloid-β protein is present in the cells of Alzheimer's disease, the amyloid-β protein binds to the magnetic bead. However, there is no amyloid-β protein binding to the magnetic bead in normal cells.

The method of diagnosing Alzheimer's disease using the giant magnetoresistance sensor according to one exemplary embodiment of the present invention includes arranging a cell to be diagnosed on the giant magnetoresistance sensor.

The giant magnetoresistance sensor may use an anisotropic magnetoresistance thin film, a giant magnetoresistance thin film, a tunnel-type magnetoresistance thin film, etc., and the giant magnetoresistance thin film used herein includes a spin valve thin film in which a great change in resistance of 5 to 10% may be realized even in a small magnetic field. The giant magnetoresistance sensor may include a free layer, a spacer, a pinned layer and a pinning layer, and may be in a cross or rod shape.

Also, the giant magnetoresistance sensor may be passivated with an oxide or nitride thin-film layer, and the cell arranged on the giant magnetoresistance sensor is preferably a brain cell, an olfactory cell, a taste cell or a visual cell, in which the amyloid-β protein can accumulate.

The method of diagnosing Alzheimer's disease using the giant magnetoresistance sensor according to one exemplary embodiment of the present invention includes disposing the pretreated magnetic bead on (above) the cell to be diagnosed, and applying an external magnetic field to magnetize the pretreated magnetic bead.

The pretreated magnetic bead is magnetized by applying the external magnetic field vertically or horizontally with respect to the surface of the giant magnetoresistance sensor, and generates a weak stray field therefrom due to the magnetization.

In the method of diagnosing Alzheimer's disease using the giant magnetoresistance sensor according to one exemplary embodiment of the present invention, Alzheimer's disease may be diagnosed by detecting a change in magnetic field according to the binding of the pretreated magnetic bead to the protein biomarker using the giant magnetoresistance sensor, and determining the presence of Alzheimer's disease in the cell to be diagnosed.

The giant magnetoresistance sensor may be used to sense a change in magnetic field by the stray field generated when the magnetic bead bound to the amyloid-β protein is magnetized by the external magnetic field, and detect the presence of the amyloid-β protein. Therefore, as an applied voltage and an input voltage supplied to the giant magnetoresistance sensor are changed, the presence of Alzheimer's disease may be determined by a magnetic resistance value of the giant magnetoresistance sensor. In this case, as the amyloid-β protein is increased in number, the bound magnetic bead is also increased in number. Therefore, the magnetic resistance value is linearly changed.

Also, the present invention provides a magnetic bead-polyprotein complex for diagnosing Alzheimer's disease, characterized in that a magnetic bead is coated with streptavidin, biotin binds to the streptavidin, and a linker is bound to the biotin so that the biotin binds to a protein biomarker.

The protein biomarker is preferably an amyloid-β protein, and the linker is preferably poly(ethylene glycol).

Furthermore, the present invention provides a method of preparing a magnetic bead-polyprotein complex for diagnosing Alzheimer's disease, characterized in that the method includes coating a magnetic bead with streptavidin; binding biotin to the streptavidin-coated magnetic bead; and binding a linker for binding to a protein biomarker to the biotin.

### Example 1

Pretreatment of Magnetic Bead (S100)

In order to sense a protein biomarker that causes Alzheimer's disease, a magnetic bead having a diameter of approximately 100 nm was pretreated. In the pretreatment of the magnetic bead, the magnetic bead was coated with streptavidin, the streptavidin bound to a biotin, and the biotin was bound to a linker, poly(ethylene glycol), so that the biotin could bind to a biomarker for the amyloid-β protein. The pretreatment allowed the magnetic bead to selectively bind to the amyloid-β protein. Here, since the amyloid-β protein was included in the cell of Alzheimer's disease, the amyloid-β protein bound to the magnetic bead. However, there is no amyloid-β protein biding to the magnetic bead in normal cells.

Arrangement of Cell to be Diagnosed (S110)

A brain cell in which the amyloid-β protein can accumulate was arranged on one upper surface of a giant magnetoresistance sensor having a spin valve structure and passivated with an oxide thin film layer. Here, the giant magnetoresistance sensor used a rod-shaped sensor including a free layer, a spacer, a pinned layer and a pinning layer.

Magnetization of Magnetic Bead (S120)

The pretreated magnetic bead was disposed on the giant magnetoresistance sensor, and an external magnetic field was applied to the pretreated magnetic bead. The external magnetic field was applied to the pretreated magnetic bead vertically with respect to a surface of the giant magnetoresistance sensor, and a weak stray field was generated from the magnetic bead magnetized by the external magnetic field.

Determination of Presence of Alzheimer's Disease (S130)

A change in magnetic field by a stray field which was generated when the magnetic bead bound to the amyloid-β protein was magnetized by an external magnetic field might be sensed using the giant magnetoresistance sensor. Then, Alzheimer's disease was diagnosed by comparing a magnetic resistance value obtained from the normal cells with a magnetic resistance value obtained from the Alzheimer's diseased cells. In this case, as the amyloid-β protein was increased in number, the bound magnetic bead was also increased in number. Therefore, the magnetic resistance value was linearly changed.

### Example 2

After a magnetic bead having a diameter of 100 nm was coated with streptavidin (S200), a biotin was bound to the streptavidin (S210), and poly(ethylene glycol) was bound to the biotin so that an amyloid-β protein could bind to the biotin (S220), thereby preparing a magnetic bead-polyprotein complex for diagnosing Alzheimer's disease.

The present invention will be described in detail with reference to the accompanying drawings, as follows. Here, detailed descriptions of known components and their related configurations according to the exemplary embodiments of the present invention are omitted for clarity since they are judged to obscure the gist of the present invention. However, the exemplary embodiments of the present invention are considered to be provided for the purpose of better understanding of the present invention as apparent to those skilled in the art. Therefore, it should be understood that the shapes and sizes of the components in the drawings may be enlarged and exaggerated for more clear description of the present invention.

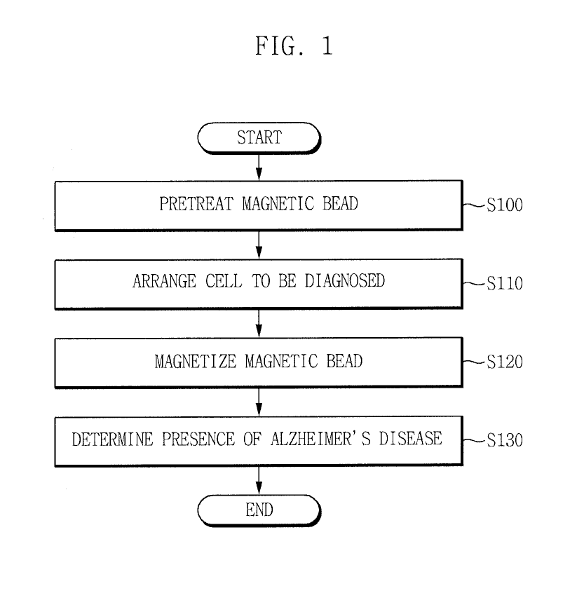

FIG. 1 is a flowchart illustrating a method of diagnosing Alzheimer's disease using a giant magnetoresistance sensor according to one exemplary embodiment of the present invention.

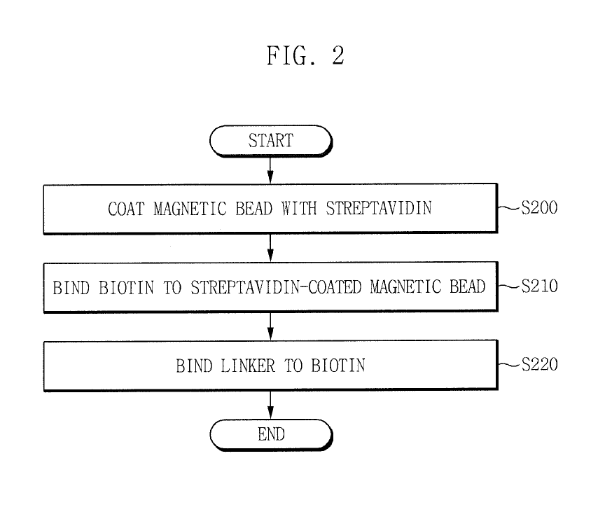

FIG. 2 is a flowchart illustrating a method of preparing a magnetic bead-polyprotein complex for diagnosing Alzheimer's disease according to one exemplary embodiment of the present invention.

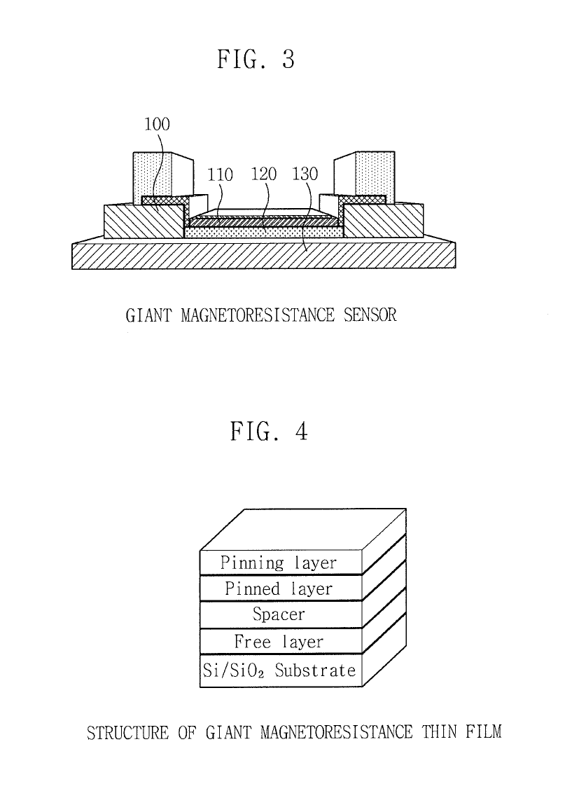

FIG. 3 is a schematic view illustrating a giant magnetoresistance sensor used to diagnose Alzheimer's disease according to one exemplary embodiment of the present invention.

Referring to FIG. 3, a layer of a giant magnetoresistance (GMR) sensor 120 is deposited on a substrate 130, a passivation layer 110 formed of an oxide is formed on the giant magnetoresistance sensor 120, and electrodes 100 formed of tantalum (Ta)/gold (Au) are formed in both sides of the giant magnetoresistance sensor 120.

FIG. 4 is a schematic view illustrating a structure of a giant magnetoresistance thin film used to diagnose Alzheimer's disease according to one exemplary embodiment of the present invention.

Referring to FIG. 4, the giant magnetoresistance sensor used in the present invention has a spin valve structure including a free layer, a spacer, a pinned layer and a pinning layer.

Since such a spin valve structure has an advantage in that a great change in resistance of 5 to 10% may be realized even in a small magnetic field, it is desirable to measure a magnetic resistance value of a magnetic particle bound to the amyloid-β protein using the giant magnetoresistance sensor.

The giant magnetoresistance sensor used in the present invention may measure a change in external magnetic field by a weak stray field generated from spherical a magnetic particle having a diameter of several tens of nanometers to several micrometers, and may display such a property of the change in resistance of the giant magnetoresistance sensor by the change in external magnetic field as an electrical output signal.

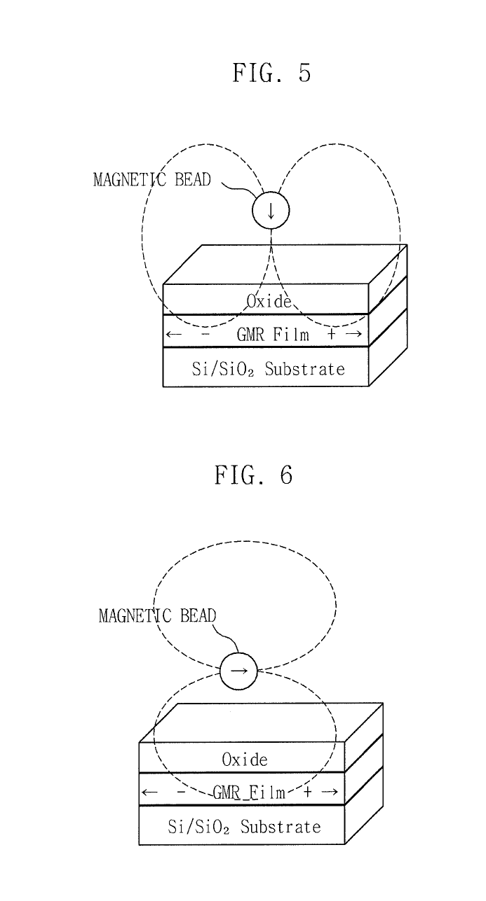

FIGS. 5 and 6 are schematic views illustrating a magnetic field direction of a magnetic bead when the magnetic bead is magnetized vertically or horizontally with respect to a surface of the giant magnetoresistance sensor.

Referring to FIGS. 5 and 6, an externally applied magnetic field may be applied vertically or horizontally with respect to a surface of the giant magnetoresistance sensor. Also, the magnetic bead may be magnetized vertically or horizontally with respect to the surface of the giant magnetoresistance sensor, as shown in FIG. 2. In this case, since a stray field generated from the magnetized magnetic bead affected magnetization of the giant magnetoresistance sensor, a resistance property of the giant magnetoresistance sensor is changed, and thus properties in output voltage by the magnetic bead are also changed.

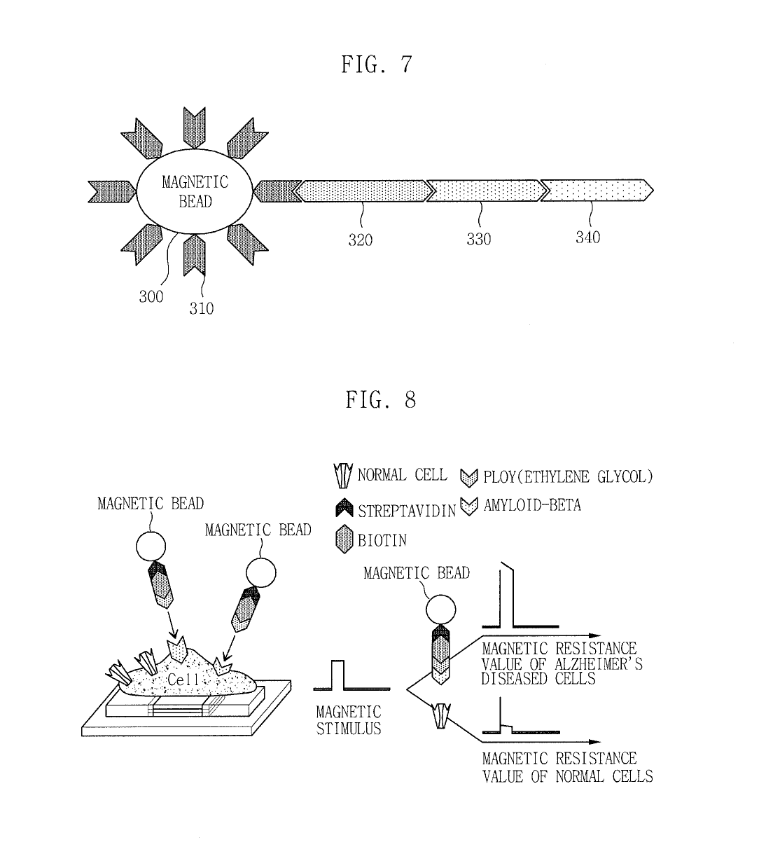

FIG. 7 is a schematic view illustrating the magnetic bead-polyprotein complex for diagnosing Alzheimer's disease according to one exemplary embodiment of the present invention.

Referring to FIG. 7, a magnetic bead (or a magnetic particle) 300 used in the present invention is coated with streptavidin 310 so that an amyloid-β protein 340 is to be selectively bound to the streptavidin 310. The magnetic bead 300 coated with the streptavidin 310 to which a biotin 320 is selectively bound, and the amyloid-β protein 340 binds to the biotin 320 via a linker such as poly(ethylene glycol) 330. In this procedure, the amyloid-β protein 340 may bind to the magnetic bead 300.

In such a manner, the magnetic bead selectively binds to the amyloid-β protein. Therefore, while cells including the amyloid-β protein which causes Alzheimer's disease bind to such magnetic beads, there is no amyloid-β protein binding to the magnetic beads in normal cells.

FIG. 8 is a schematic view illustrating a method of diagnosing Alzheimer's disease using a giant magnetoresistance sensor according to another exemplary embodiment of the present invention.

Referring to FIG. 8, cells to be diagnosed are arranged on a giant magnetoresistance sensor, and a magnetic bead is magnetized by applying an external magnetic field to the magnetic bead. Then, the giant magnetoresistance sensor may detect the presence of the amyloid-β protein by sensing a change in magnetic field by a stray current generated from the magnetic bead. Therefore, the giant magnetoresistance sensor may determine the presence of Alzheimer's disease by comparing a magnetic resistance value obtained from the normal cells with a magnetic resistance value obtained from the Alzheimer's diseased cells. Since the magnetic bead is increased in number as the amyloid-β protein is increased in number, the magnetic resistance value is linearly changed. As a result, by measuring the change in magnetic resistance value using the magnetic bead bound to the amyloid-β protein, Alzheimer's disease may be diagnosed at an early stage and development of Alzheimer's disease may also be diagnosed.

According to the present invention, Alzheimer's disease can be diagnosed more easily and simply than the conventional methods using a fluorescent material, genetic analysis or comparison, and the magnetic bead-polyprotein complex of the present invention can be commercially available as a biosensor for diagnosing Alzheimer's disease.

As described above, a method of diagnosing Alzheimer's disease using a giant magnetoresistance sensor according to the present invention can be applied to easy and simple diagnosis of Alzheimer's disease using the giant magnetoresistance sensor rather than conventional fluorescent materials or genetic analyses.

Also, the method of diagnosing Alzheimer's disease using the giant magnetoresistance sensor according to the exemplary embodiment of the present invention can be useful in monitoring and treating Alzheimer's disease since the method can provide mass-production of a biosensor for diagnosing Alzheimer's disease.

While the example embodiments of the present invention and their advantages have been described in detail, it should be understood that various changes, substitutions and alterations may be made herein without departing from the scope of the invention.

What is claimed is:

1. A method of diagnosing Alzheimer's disease using a giant magnetoresistance sensor, the method comprising:
pretreating a magnetic bead so that the magnetic bead is to be bound to a protein biomarker causing Alzheimer's disease;
arranging a cell to be diagnosed on the giant magnetoresistance sensor;
magnetizing the pretreated magnetic bead by applying an external magnetic field; and
determining the presence of Alzheimer's disease in the cell to be diagnosed by detecting a change in magnetic field according to the binding of the pretreated magnetic bead to the protein biomarker using the giant magnetoresistance sensor.

2. The method of claim 1, wherein the change in magnetic field is caused by a stray field generated from the magnetic bead when the magnetic bead binds to the protein biomarker.

3. The method of claim 1, wherein the protein biomarker is an amyloid-β protein.

4. The method of claim 1, wherein the magnetic bead has a diameter of 50 nm to 5 μm.

5. The method of claim 1, wherein the pretreatment is performed by coating the magnetic bead with streptavidin, binding biotin to the streptavidin-coated magnetic bead and binding poly(ethylene glycol) to the biotin.

6. The method of claim 1, wherein the giant magnetoresistance sensor is one selected from the group consisting of an anisotropic magnetoresistance thin film, a giant magnetoresistance thin film and a tunnel-type magnetoresistance thin film.

7. The method of claim 1, wherein the giant magnetoresistance sensor comprises a free layer, a spacer, a pinned layer and a pinning layer.

8. The method of claim 1, wherein the giant magnetoresistance sensor is formed in a cross or rod shape.

9. The method of claim 1, wherein the giant magnetoresistance sensor is passivated with an oxide or nitride thin-film layer.

10. The method of claim 1, wherein the cell to be diagnosed is a brain cell, olfactory cell, taste cell or visual cell, in which the amyloid-β protein accumulates.

11. The method of claim 1, wherein the magnetization is performed by applying the external magnetic field vertically or horizontally with respect to the surface of the giant magnetoresistance sensor.

12. A magnetic bead-polyprotein complex for diagnosing Alzheimer's disease, in which a magnetic bead is coated with streptavidin, biotin binds to the streptavidin, and a linker for binding to a protein biomarker binds to the biotin.

13. The magnetic bead-polyprotein complex of claim 12, wherein the protein biomarker is an amyloid-β protein.

14. The magnetic bead-polyprotein complex of claim 12, wherein the linker is poly(ethylene glycol).

15. A method of preparing a magnetic bead-polyprotein complex for diagnosing Alzheimer's disease, the method comprising:
coating a magnetic bead with streptavidin;
binding biotin to the streptavidin-coated magnetic bead; and
binding a linker to the biotin,
wherein the linker is to be bound to a protein biomarker.
